(19) United States
(12) Patent Application Publication
(10) Pub. No.: US 2024/0283275 A1
(43) Pub. Date: Aug. 22, 2024

(54) WEARABLE SENSOR TERMINAL

(71) Applicant: Nippon Telegraph and Telephone Corporation, Tokyo (JP)

(72) Inventors: Akio Tokura, Tokyo (JP); Toshihiko Kondo, Tokyo (JP)

(21) Appl. No.: 18/569,922

(22) PCT Filed: Jun. 24, 2021
(86) PCT No.: PCT/JP2021/023951

§ 371 Date: Dec. 13, 2023

(51) Int. Cl. H02J 7/00; A61B 5/00; H04W 4/38

(52) U.S. Cl. CPC H02J 7/007182; H02J 7/00306; H02J 7/0068; A61B 5/6802; H02J 2207/20; H04W 4/38

(57) ABSTRACT

An embodiment wearable sensor terminal includes a secondary battery, a charging IC that performs fast charging of the secondary battery when the voltage of the secondary battery is equal to or higher than a threshold voltage, a wireless communication module, an LDO regulator that converts the voltage of the secondary battery and supplies the wireless communication module with the voltage, and a protection circuit that stops the supply of the power supply voltage from the LDO regulator to the wireless communication module when the voltage of the secondary battery is lower than the threshold voltage at the time of charging.

### CROSS-REFERENCE TO RELATED APPLICATIONS

This application is a national phase entry of PCT Application No. PCT/JP2021/023951, filed on Jun. 24, 2021, which application is hereby incorporated herein by reference.

### TECHNICAL FIELD

The present invention relates to a wearable sensor terminal, particularly a wearable sensor terminal that measures biological information and environmental information on the vicinity of a living body using a secondary battery.

### BACKGROUND

It is important to monitor biological information and environmental information for physical condition management such as prevention of heat stroke under heat. For example, in a heat index meter conventionally used for preventing heat stroke, a heat index is obtained by measuring a black-bulb temperature, a wet-bulb temperature, and a dry-bulb temperature, and a method for guiding action such as avoiding going out or hard work in a case where the heat index is relatively high is used (refer to Non Patent Literature 1).

However, the heat load actually received by each person greatly depends on the local environment. For example, the environment varies greatly depending on where each person is, such as outdoors or indoors, sunlit or shaded, and on lawn or on concrete. Moreover, even in the same place, the influence of radiation from the ground is greatly different between a tall adult and a short child, for example. Furthermore, the environment of a human body greatly changes depending on clothes worn, an exercise state, a sweating state, and the like.

Thus, in order to monitor biological information particularly in accordance with the environment in the vicinity of the human body, a method of carrying and wearing a biological sensor and an environmental sensor is conceivable. Therefore, there is known a small wearable sensor terminal that can be attached to clothes, underwear, or the like of an individual to measure the immediate environment of the clothes, the environment inside the clothes, and biological information for each individual (refer to Patent Literature 1).

FIG. 7A is a front view of a conventional wearable sensor terminal, and FIG. 7B is a rear view of the conventional wearable sensor terminal. A conventional wearable sensor terminal 100 is configured with a resin housing 101 made of resin such as acrylonitrile butadiene styrene (ABS) or polycarbonate, for example. Since the wearable sensor terminal 100 is small and lightweight, it is designed to be worn without imposing a burden on the wearer.

The wearable sensor terminal 100 includes a plurality of sensors. Specifically, a snap 102 on the back surface serves as an electrode of an electrocardiogramell as a connection portion to dedicated clothes or underwear. The electrocardiogram waveform of the wearer can be acquired by connecting the snap 102 with wear or a belt including an electrode in contact with the body of the wearer or connecting the snap 102 with a patch electrode to measure the biological potential through the snap 102. An analog front end circuit 109 for processing an electrocardiogram waveform may be provided inside the wearable sensor terminal 100.

Moreover, the wearable sensor terminal 100 includes a temperature/humidity sensor 103 inside the right end as viewed from the front side. The temperature/humidity sensor 103 enables measurement of the temperature and the humidity in the vicinity of the wearer, particularly the temperature and the humidity in clothes in a case where the wearable sensor terminal 100 is worn in the clothes. A ventilation hole 104 is formed around the temperature/humidity sensor 103. This facilitates access to the environment and enables quick response to temperature and humidity.

Furthermore, the wearable sensor terminal 100 includes an acceleration/angular velocity sensor 105 therein. The acceleration/angular velocity sensor 105 makes it possible to grasp the amount of activity through analysis of the movement of the wearer. The wearable sensor terminal 100 includes an LED 106 on the upper left when viewed from the front side. The LED 106 enables display of a terminal status including ON/OFF of a power supply. The power supply of the wearable sensor terminal 100 is turned on/off by pressing a power button 107 located on the upper right when viewed from the front side. The wearable sensor terminal 100 includes a secondary battery (rechargeable battery) therein. By charging the secondary battery through a universal serial bus (USB) connector 108 located on the back surface, the secondary battery can be repeatedly used without battery replacement.

Various kinds of measured data are transmitted to a smartphone, an Internet of Things (IoT) gate, a personal computer (PC), or the like, which is a data receiving terminal, via a wireless communication device mounted inside the wearable sensor terminal 100. The data may be further retained in a cloud server or the like from the data receiving terminal via the Internet. In the case of communication from the wearable sensor terminal 100 to the data receiving terminal, a short-distance wireless communication standard for communication to the vicinity of the sensor is often adopted instead of wireless for long-distance communication used in mobile phones, in order to reduce power consumption of wireless communication. As a short-distance wireless communication standard, Bluetooth (registered trademark) low energy (BLE) or the like is often used.

FIG. 8 is a diagram illustrating a circuit of a power supply unit of the wearable sensor terminal 100. A DC voltage of 5 V is supplied from the outside via the USB connector 108. A charging integrated circuit (IC) 110 that controls a charging current supplies the secondary battery 111 with an appropriate charging current to charge the secondary battery 111. The charging IC 110 performs low-speed charging (pre-charge) of performing charging with a low current until the voltage VBAT of the secondary battery 111 reaches a predetermined voltage, and performs fast charging (first-charge) of performing charging with a relatively large current after the voltage VBAT reaches the predetermined voltage.

The secondary battery 111 is connected with a protection IC 112 for preventing an unintended operation. The protection IC 112 prevents overcharging, excessive current consumption, and overdischarging of the secondary battery 111. For example, when the voltage VBAT of the secondary battery 111 becomes equal to or lower than a predetermined lower limit voltage, the protection IC 112 stops current supply (for example, makes the current equal to or lower than the order of μA) to prevent overdischarging of the secondary battery 111.

The secondary battery 111 is connected with a device or a control device of the wearable sensor terminal 100 via a low dropout (LDO) regulator 113 that suppresses voltage fluctuation. The LDO regulator 113 includes an IN terminal for voltage input, an OUT terminal for voltage output, a ground (GND) terminal, a Vset terminal for setting a supply voltage value, and an EN (enable) terminal for controlling output. The LDO regulator 113 outputs a voltage corresponding to an output selection signal inputted to the Vset terminal from the OUT terminal.

Moreover, the LDO regulator 113 has a switch function of turning on/off the output according to the input of the EN terminal. The LDO regulator 113 supplies each device of the wearable sensor terminal 100 with a voltage from the OUT terminal when the EN terminal is in a high voltage state (high), and stops the voltage supply from the OUT terminal when the EN terminal is in a low voltage state (Low).

The voltage from the LDO regulator 113 is also supplied to the control device of the wearable sensor terminal 100. As a control device, there is also a wearable device including a dedicated microprocessor (MPU: Microprocessing Unit). However, at present, with the progress of high functionality and multi-functionality of communication devices, a wireless communication module 114 with a signal processor function is increasingly adopted in order to achieve space saving, low power consumption, and low price. An example of the wireless communication module 114 with a signal processor function is a BLE communication module with a microprocessor function. The BLE communication module has a control function of the entire wearable sensor terminal in addition to an original function of BLE communication.

In the conventional wearable sensor terminal 100, the following problems may occur. The wireless communication module 114 is activated at a predetermined power supply voltage or higher, and operates according to a program of system software called firmware. However, even though the voltage is equal to or lower than a predetermined power supply voltage, a part of the internal communication function may start to be activated, so that the current starts to be consumed while the control of the wireless communication module 114 is not effective, and the current consumption continues.

FIG. 9 illustrates an example of a power supply unit in the wireless communication module 114. The power supply voltage supplied from the LDO regulator 113 is sent to a DC/DC converter 115 in the wireless communication module 114. The DC/DC converter 115 supplies each unit of the module with a predetermined voltage. That is, when a power supply voltage equal to or higher than the predetermined voltage is supplied to a VDD terminal of the wireless communication module 114, power may be supplied from the DC/DC converter 115 to each unit of the module.

When the control device and the communication device are separate, the operation of the communication device can be controlled by the control device. Alternatively, it is possible to take measures such as stopping voltage supply to the communication device in a state where the control device is not activated. However, in a case where the wireless communication module 114 also serves as the control device as in the example of FIG. 8, it is impossible to stop the phenomenon that occurs in a state where the control device is not activated.

The problem of current consumption that occurs when the control device is not activated may occur at a voltage equal to or lower than the activation voltage of the wireless communication module 114. Here, since the voltage VBAT of the secondary battery 111 is low in a state where the control device is not activated, when there is no voltage supply from the USB connector 108 and the secondary battery 111 is not charged, the overdischarging prevention function by the protection IC 112 works and the voltage supply to the LDO regulator 113 is stopped, so that no problem occurs.

However, when the voltage is supplied from the USB connector 108, charging of the secondary battery 111 is started, and low-speed charging is performed, the voltage supply to the LDO regulator 113 through the charging IC 110 is restored, and the power supply voltage is also supplied to the wireless communication module 114. In the low-speed charging state, since the total amount of the charging current supplied from the charging IC 110 to the system is small, most of the supplied current is consumed by the wireless communication module 114 as it is, the voltage of the secondary battery 111 does not increase, and the charging never ends. In such a state, the wearable sensor terminal 100 does not normally function.

Although the wireless communication function can be always turned off during charging of the secondary battery 111, it is desirable that a function such as communication can be used even during charging as long as the voltage of the secondary battery 111 is in a normal range.

### CITATION LIST

### Non Patent Literature

Non Patent Literature 1: JuYoun Kwon, Ken Parsons, “Evaluation of the Wet Bulb Globe Temperature (WBGT) Index for Digital Fashion Application in Outdoor Environments”, Journal of the Ergonomics Society of Korea, 36, pp. 23-36, 2017.

### SUMMARY

### Technical Problem

Embodiments of the present invention can solve the above problems, and an object thereof is to solve a problem that a charging current is consumed by a wireless communication device or the like during charging of a secondary battery and charging becomes impossible, and realize a wearable sensor terminal that always operates normally so as to enable simple, stable, and accurate measurement of biological information and environmental information on the vicinity of a living body.

### Solution to Problem

An embodiment of the present invention includes: a sensor configured to measure at least one of biological information or environmental information on the vicinity of a living body; a wireless communication device configured to wirelessly transmit information measured by the sensor to the outside; a secondary battery; a charging control device configured to perform fast charging of the secondary battery when a voltage of the secondary battery is equal to or higher than a first threshold voltage; a battery protection device configured to prevent overdischarging of the secondary battery; a regulator configured to convert the voltage of the secondary battery and supply the wireless communication device with the voltage; and a protection circuit configured to stop supply of a power supply voltage from the regulator to the wireless communication device when the voltage of the secondary battery is lower than the first threshold voltage at the time of charging.

Moreover, a configuration example of a wearable sensor terminal according to an embodiment of the present invention further includes a first resistor having one end connected with a positive electrode of the secondary battery and an input terminal of the regulator, and the other end connected with a control terminal of the regulator, in which the protection circuit includes: a second resistor having one end connected with a connection point between the first resistor and the control terminal of the regulator; and a digital transistor having a control input terminal to which a voltage from an external power supply for charging the secondary battery is inputted, a collector terminal connected with the other end of the second resistor, and an emitter terminal connected with the ground.

Moreover, a configuration example of a wearable sensor terminal according to an embodiment of the present invention further includes a resistor having one end connected with a positive electrode of the secondary battery and an input terminal of the regulator, and the other end connected with a control terminal of the regulator, in which the protection circuit includes: a comparator configured to compare a reference voltage with a voltage of the secondary battery; and a digital transistor having a control input terminal to which an output voltage of the comparator is inputted, a collector terminal connected with a connection point between the resistor and the control terminal of the regulator, and an emitter terminal connected with the ground.

Moreover, in a configuration example of a wearable sensor terminal according to an embodiment of the present invention, the protection circuit further includes a voltage dividing circuit configured to generate the reference voltage from an external power supply for charging the secondary battery.

Moreover, in a configuration example of a wearable sensor terminal according to an embodiment of the present invention, the regulator supplies the wireless communication device with the power supply voltage when the voltage of the control terminal is equal to or higher than a second threshold voltage, and stops supply of the power supply voltage to the wireless communication device when the voltage of the control terminal is equal to or lower than a third threshold voltage that is lower than the second threshold voltage.

Moreover, a configuration example of a wearable sensor terminal according to an embodiment of the present invention includes at least one of an electrocardiogram sensor, an acceleration sensor, an angular velocity sensor, or a temperature/humidity sensor as the sensor.

Moreover, in a configuration example of a wearable sensor terminal according to an embodiment of the present invention, the wireless communication device is a BLE communication module.

### Advantageous Effects of Embodiments of the Invention

According to embodiments of the present invention, by providing the protection circuit, the power supply voltage is not supplied from the regulator to the wireless communication device when the voltage of the secondary battery is lower than the first threshold voltage even during charging, so that a phenomenon that all or most of the charging current is consumed by the wireless communication device and charging is not performed does not occur. Accordingly, the wearable sensor terminal can be operated normally. Furthermore, in a case where the voltage of the secondary battery is sufficiently high, the wireless communication function can be used while performing charging. Therefore, it is possible to simply, stably, and accurately measure biological information and environmental information on the vicinity of a living body.

### BRIEF DESCRIPTION OF THE DRAWINGS

FIG. 1 is a diagram illustrating a circuit of a power supply unit of a wearable sensor terminal according to a first embodiment of the present invention.

FIG. 2 is a diagram for explaining an operation of a power supply unit of a wearable sensor terminal according to a second embodiment of the present invention.

FIG. 3 is a diagram for explaining an operation of a power supply unit of a wearable sensor terminal according to the second embodiment of the present invention.

FIG. 4 is a diagram illustrating a circuit of a power supply unit of a wearable sensor terminal according to a third embodiment of the present invention.

FIG. 5 is a diagram for explaining an operation of a power supply unit of a wearable sensor terminal according to a fourth embodiment of the present invention.

FIG. 6 is a diagram for explaining an operation of a power supply unit of a wearable sensor terminal according to the fourth embodiment of the present invention.

FIG. 7A is a front view of a conventional wearable sensor terminal.

FIG. 7B is a rear view of the conventional wearable sensor terminal.

FIG. 8 is a diagram illustrating a circuit of a power supply unit of a conventional wearable sensor terminal.

FIG. 9 is a diagram illustrating a circuit of a power supply unit in a wireless communication module of a conventional wearable sensor terminal.

### DETAILED DESCRIPTION OF ILLUSTRATIVE EMBODIMENTS

### Principle of Embodiments of the Invention

A phenomenon that a failure occurs in the charging function of the wearable sensor terminal occurs because the LDO regulator outputs a voltage to the wireless communication module during low-speed charging of the secondary battery. As a result of detailed investigation, it has been found that the current supplied from the charging IC is large during fast charging of the secondary battery, and therefore, even if a part of the current is consumed by the wireless communication module, the remaining current is supplied to the secondary battery, and the charging function can be normally maintained.

Accordingly, it is found that the output of the LDO regulator needs to be stopped only during low-speed charging of the secondary battery, in order to solve the above problems. There has been no clear report case on the same type of failure in the wearable terminal before the embodiments of the present invention, and accordingly, the method of embodiments of the present invention for solving problems and the device configuration of embodiments of the present invention are not obvious or known to those skilled in the art. As a result of various kinds of verifications, the inventor has found that it is a drastic solution to connect a control terminal of an LDO regulator with a protection circuit that stops the output of the LDO regulator when the voltage of the secondary battery is lower than a threshold voltage at which low-speed charging is switched to fast charging.

### First Embodiment

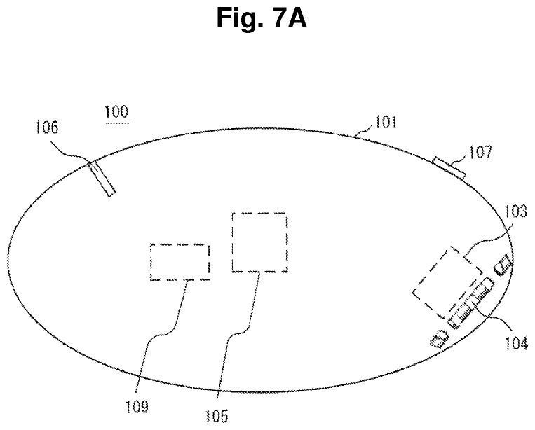

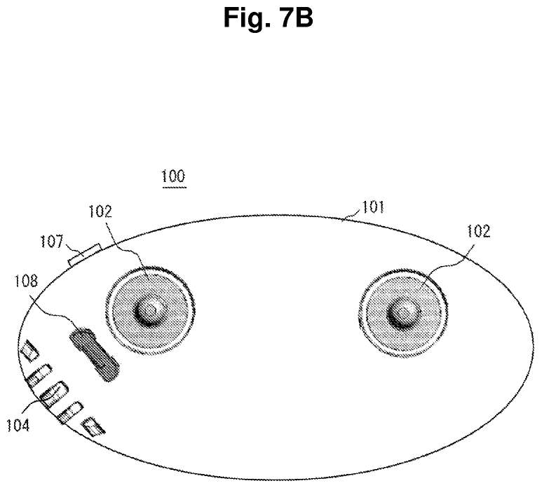

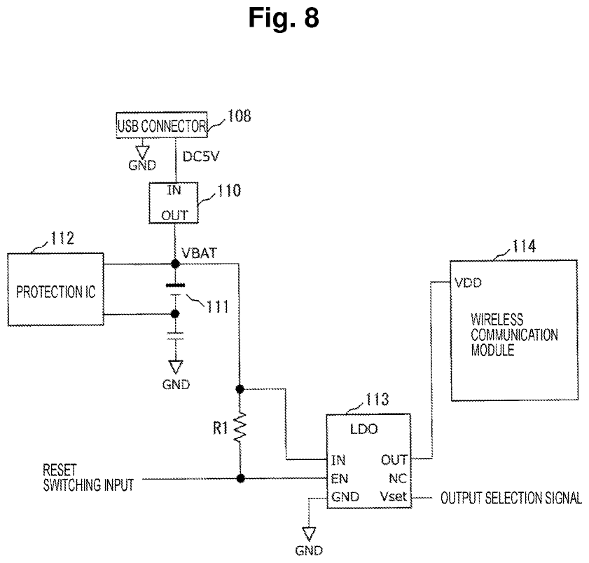

Hereinafter, preferred embodiments of the present invention will be described in detail. Also in the present embodiment, since the configuration of the wearable sensor terminal except the power supply unit is similar to that of the conventional wearable sensor terminal, the description will be made using reference numerals in FIGS. 7A, 7B, and 8. Similarly to the conventional wearable sensor terminal, a wireless communication module 114 (wireless communication device) in the wearable sensor terminal transmits, to a data receiving terminal, measured data (biological information) of the electrocardiogram of the wearer (living body) measured by an electrocardiogram sensor (a snap 102 and an analog front end circuit 109), measured data (environmental information) of the temperature and the humidity measured by a temperature/humidity sensor 103, and measured data (environmental information) of the acceleration and the angular velocity measured by an acceleration/angular velocity sensor 105. The wireless communication module 114 is operated by a voltage supplied from an LDO regulator 113. Although the acceleration and the angular velocity are measured by the acceleration/angular velocity sensor 105 in this example, an acceleration sensor and an angular velocity sensor may be separate.

Note that, although both the biological information and the environmental information are measured in the present embodiment, embodiments of the present invention can be applied to a wearable sensor terminal that measures at least one of the biological information or the environmental information.

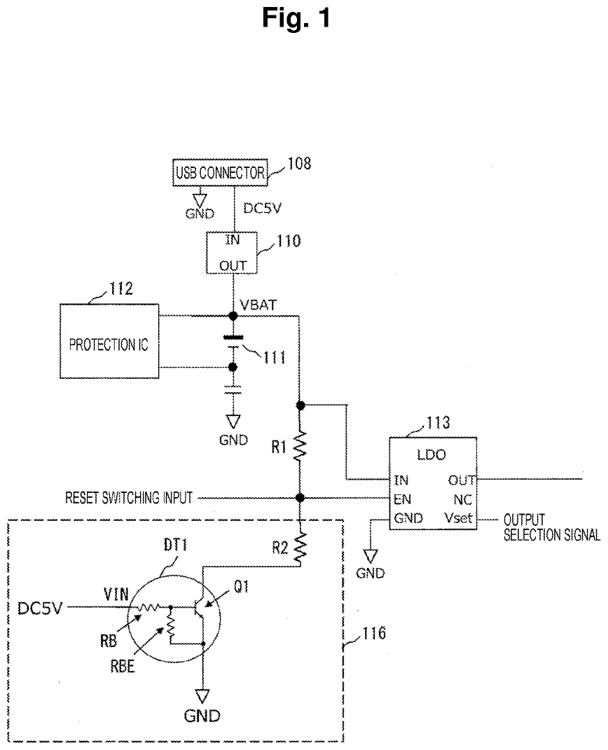

FIG. 1 is a diagram illustrating a circuit of a power supply unit of a wearable sensor terminal according to the present embodiment. The configuration of a power supply unit of the present embodiment is obtained by adding a protection circuit 116 to the power supply unit of the conventional wearable sensor terminal illustrated in FIG. 8.

The protection circuit 116 is configured with a digital transistor DT1 and a resistor R2. In the digital transistor DT1, a resistor RB is inserted between a control input terminal VIN of the digital transistor DT1 and a base terminal of a bipolar transistor Q1, and a resistor RBE is inserted between the base terminal and an emitter terminal of the bipolar transistor Q1.

Similarly to a conventional power supply unit, there is provided a resistor R1 having one end connected with a positive electrode of a secondary battery 111 and an IN terminal (input terminal) of the LDO regulator 113, and the other end connected with an EN terminal (control terminal) of the LDO regulator 113. The resistor R2 of the protection circuit 116 has one end connected with a connection point between the resistor R1 and the EN terminal of the LDO regulator 113, and the other end connected with a collector terminal of the digital transistor DT1. The control input terminal VIN of the digital transistor DT1 is supplied with a DC voltage of 5 V from an external power supply connected with a USB connector 108 of the wearable sensor terminal. An emitter terminal of the digital transistor DT1 is connected with the ground (GND).

When an external power supply is connected with the USB connector 108 and a DC voltage of 5 V is supplied to the control input terminal VIN of the digital transistor DT1, the digital transistor DT1 is turned on. As a result, the collector terminal of the digital transistor DT1 and the ground are connected, and the voltage at the connection point between the two resistors R1 and R2 lowers.

On the other hand, when the external power supply is disconnected from the USB connector 108 and the voltage supply to the control input terminal VIN of the digital transistor DT1 is stopped, the digital transistor DT1 is turned off. As a result, current does not flow through the resistor R2, and the voltage at the connection point between the two resistors R1 and R2, that is, the voltage of the EN terminal of the LDO regulator 113, becomes the voltage VBAT of the secondary battery 111.

As described above, since the protection circuit 116 is provided, the input voltage of the EN terminal of the LDO regulator 113 can be lowered only at the time of charging when an external power supply is connected with the USB connector 108 of the wearable sensor terminal. How much the input voltage of the EN terminal of the LDO regulator 113 is lowered from the voltage VBAT of the secondary battery 111 can be arbitrarily set by the voltage division ratio of the two resistors R1 and R2.

Thus, the voltage division ratio of the resistors R1 and R2 is designed such that the EN terminal of the LDO regulator 113 is in a high voltage state (high) when the voltage VBAT of the secondary battery 111 is equal to or higher than the voltage value (first threshold voltage) at which the low-speed charging is switched to the fast charging. As a result, the LDO regulator 113 supplies each device of the wearable sensor terminal with a voltage from the OUT terminal only when the charging IC 110 is performing fast charging of the secondary battery 111. On the other hand, when the charging IC 110 is performing low-speed charging of the secondary battery 111, the EN terminal of the LDO regulator 113 is in a low voltage state (Low) because the voltage VBAT of the secondary battery 111 is low, and therefore, the LDO regulator 113 stops the voltage supply from the OUT terminal.

As described above, in the present embodiment, the voltage supply from the LDO regulator 113 is stopped at the time of low-speed charging of the secondary battery 111, so that the secondary battery 111 can be charged normally, and the wearable sensor terminal can be operated normally even during charging. Moreover, since the protection circuit 116 has a simple configuration, it is possible to prevent charging failure of the secondary battery 111 with an inexpensive and space-saving configuration.

### Second Embodiment

Next, a second embodiment of the present invention will be described. The present embodiment describes a specific example of the first embodiment. The outer shape, the mounted sensor type, the basic design, and the function of a wearable sensor terminal of the present embodiment are the same as those of the conventional wearable sensor terminal illustrated in FIGS. 7A and 7B. The configuration of the power supply unit is as illustrated in FIG. 1.

A resin housing 101 is formed of, for example, ABS. As the sensors, an electrocardiogram sensor (a snap 102 and an analog front end circuit 109), a temperature/humidity sensor 103, and an acceleration/angular velocity sensor 105 are mounted. The wearable sensor terminal is provided with a waterproof USB connector 108 having a micro USB type B shape. By connecting the USB connector 108 with an external power supply, a secondary battery 111 can be charged. Examples of the secondary battery 111 include a lithium ion battery. The maximum charging voltage (maximum value of voltage VBAT) is 4.2 V.

When the voltage VBAT of the secondary battery 111 becomes equal to or higher than a predetermined voltage value V₁, a charging IC 110 (charging control device) switches from low-speed charging to high-speed charging. The charging current in low-speed charging is A₁, and the charging current in high-speed charging is A₂ (A₁<A₂).

A protection IC 112 (battery protection device) prevents overcharging at a voltage VBAT of V₂ or higher, discharging due to overcurrent, and overdischarging at V₃ (V₂>V₃) or lower. For example, the protection IC 112 suppresses the discharge current of the secondary battery 111 to the order of μA or less when the voltage VBAT becomes V₃ or lower.

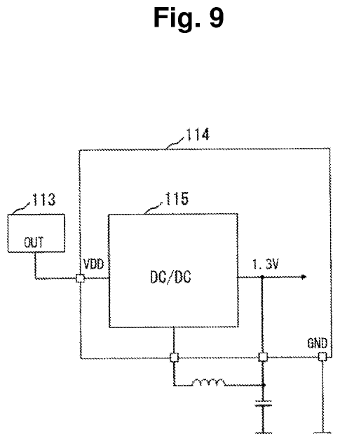

As described above, a wireless communication module 114 (BLE communication module) with a signal processor function is adopted in the wearable sensor terminal in order to achieve space saving, low power consumption, and low price. The wireless communication module 114 also controls the entire wearable sensor terminal. The power supply voltage at which the wireless communication module 114 is normally activated is V₄ to V₅ (V₄<V₅). As described with reference to FIG. 9, the power supply voltage supplied to the wireless communication module 114 is converted into a predetermined voltage by an internal DC/DC converter 115 and supplied to each unit of the module.

The output of an LDO regulator 113 is set to V₆ (V₄<V₆<V₅) in accordance with the standard value of the power supply voltage of the wireless communication module 114. The output of the LDO regulator 113 is turned on when the EN terminal is equal to or higher than V₇ (second threshold voltage), and is turned off when the EN terminal is equal to or lower than V₈ (third threshold voltage). When the EN terminal becomes equal to or lower than V₈ and the output of the LDO regulator 113 is turned off, the output of the LDO regulator 113 is not turned on until the EN terminal returns to V₇ (V₈<V₇) or higher. When the protection IC 112 functions and is in the overdischarging prevention (current supply restriction) state, the voltage supply to the LDO regulator 113 is stopped by stopping the current supply from the battery, so that the EN terminal becomes equal to or lower than V₈ and the enable input is reset.

As illustrated in FIG. 1, the power supply unit includes a protection circuit 116 in the present embodiment. At the time of charging the secondary battery 111, a DC voltage of 5 V is supplied from an external power supply to the control input terminal VIN of the digital transistor DT1 of the protection circuit 116 via the USB connector 108. The electric resistance of RA is used as the resistor R1 of the power supply unit, and the electric resistance of RB is used as the resistor R2 of the protection circuit 116.

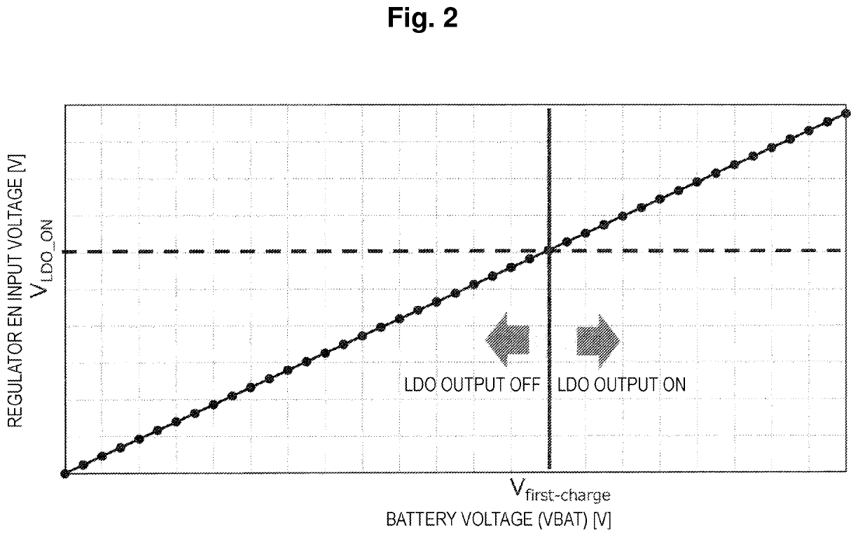

FIG. 2 is a diagram for explaining an operation of a power supply unit of the wearable sensor terminal of the present embodiment. In FIG. 2, the horizontal axis represents the voltage VBAT of the secondary battery 111, and the vertical axis represents the voltage of the EN terminal of the LDO regulator 113. V_{first-charge} is a threshold voltage (V₁) at which the charging IC 110 switches from low-speed charging to fast charging, and V_{LDO_ON} is a threshold voltage (V₇) at which the output of the LDO regulator 113 is turned on.

According to FIG. 2, the EN terminal of the LDO regulator 113 operates to be equal to or higher than V_{LDO_ON} at a voltage equal to or higher than V_{first-charge} at which the charging is reliably switched to fast charging. That is, RA and RB are set such that V₁×{RB/(RA+RB)}≤V₇ is satisfied. In this case, the EN terminal does not become equal to or higher than V_{LDO_ON} in VBAT that satisfies VBAT<V₁. Accordingly, a state where all or most of the charging current of the secondary battery 111 is consumed by the wireless communication module 114 and the secondary battery 111 cannot be charged does not occur.

In a conventional wearable sensor terminal that does not use the protection circuit 116, a charging failure of the secondary battery 111 was observed.

On the other hand, regarding a wearable sensor terminal including the protection circuit 116 of the present embodiment, no charging failure of the secondary battery 111 occurred in all the prepared terminals. In addition, as in the original goal, the communication function can be utilized while charging in a case where the voltage VBAT of the secondary battery 111 is sufficiently high.

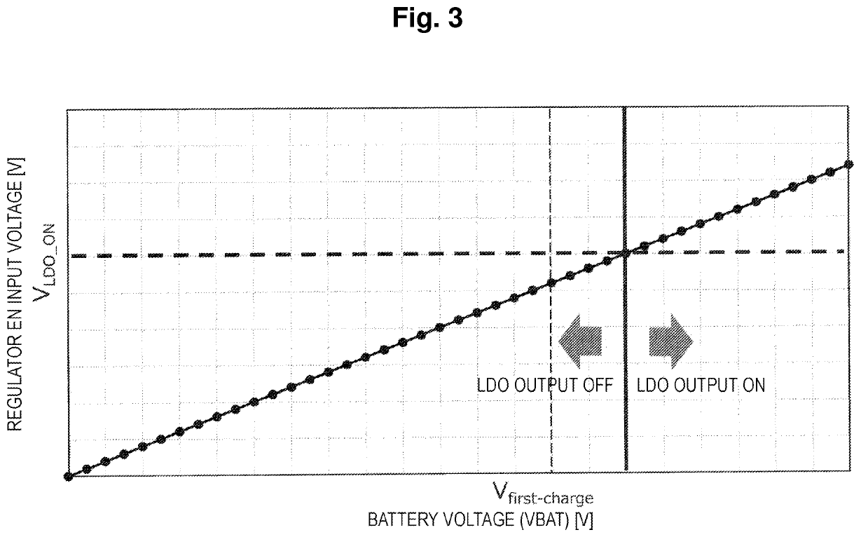

As described above, since the voltage division ratio setting of the two resistors R1 and R2 is arbitrary, it is also possible to set the voltage division ratio not to a value close to the threshold but to a value with a little more margin. FIG. 3 illustrates an operation of the power supply unit in a case where the resistor R1 is RC (RA<RC) and the resistor R2 is RD (RB>RD).

In the example of FIG. 2, it is set such that the EN terminal of the LDO regulator 113 becomes equal to or higher than the threshold voltage V_{LDO_ON}, and the output of the LDO regulator 113 it turned on when the voltage VBAT of the secondary battery 111 reaches the threshold voltage V_{first-charge}.

On the other hand, in the example of FIG. 3, the EN terminal of the LDO regulator 113 becomes equal to or higher than the threshold voltage V_{LDO_ON} when the voltage VBAT of the secondary battery 111 becomes sufficiently higher than the threshold voltage V_{first-charge}. The EN terminal becomes equal to or higher than the threshold voltage V_{LDO_ON} when the voltage VBAT is equal to or higher than V₇×{(RC/RD)+1}. Also in the example of FIG. 3, no charging failure of the secondary battery 111 was observed.

### Third Embodiment

Next, a third embodiment of the present invention will be described. The protection circuit 116 described in the first and second embodiments can be realized with an inexpensive and space-saving configuration. However, since the input value to the EN terminal of the LDO regulator 113 shows a continuous change, another device is required if it is desired to clarify the threshold or to perform an ON/OFF switching operation.

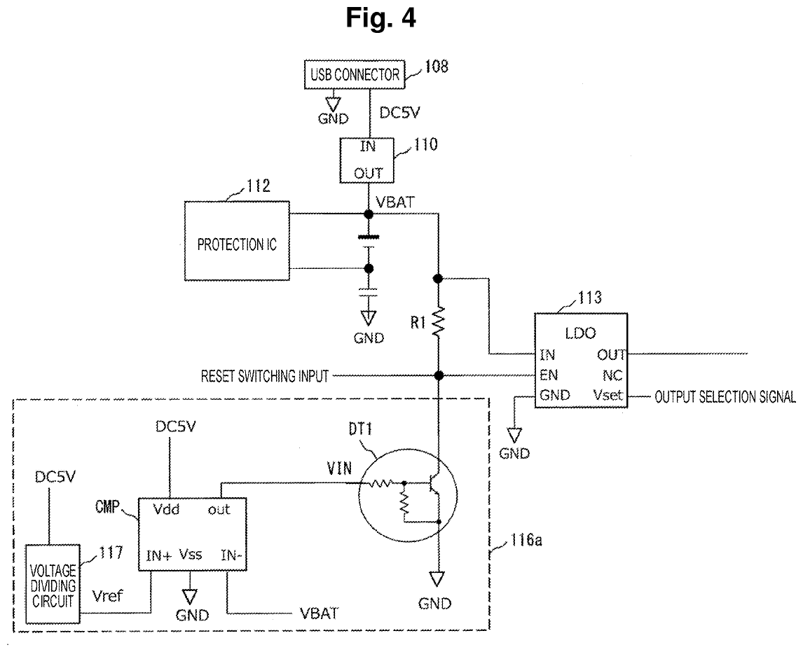

FIG. 4 is a diagram illustrating a circuit of a power supply unit of a wearable sensor terminal according to the present embodiment. The configuration of the power supply unit of the present embodiment is obtained by adding a protection circuit 116a to the power supply unit of the conventional wearable sensor terminal illustrated in FIG. 8.

The protection circuit 116a includes a digital transistor DT1, a comparator CMP, and a voltage dividing circuit 117. A collector terminal of the digital transistor DT1 is connected with a connection point between a resistor R1 and an EN terminal of an LDO regulator 113. An emitter terminal of the digital transistor DT1 is connected with the ground.

The comparator CMP is an electronic circuit that can obtain different outputs depending on the magnitude of two inputs. A reference voltage Vref is inputted to an IN+ terminal of the comparator CMP, and a voltage VBAT of a secondary battery 111 is inputted to an IN− terminal. A DC voltage of 5 V is supplied from an external power supply to a Vdd terminal of the comparator CMP via a USB connector 108, a Vss terminal is connected with the ground, and an out terminal is connected with a control input terminal VIN of the digital transistor DT1.

The comparator CMP outputs 5 V inputted to the Vdd terminal from the out terminal when the reference voltage Vref is higher than the voltage VBAT, and sets the out terminal to the ground potential when the reference voltage Vref is equal to or lower than the voltage VBAT. That is, the out terminal output takes a binary value of 5 V and the ground potential, which makes an ON/OFF switching operation.

Since an external power supply is connected with the Vdd terminal of the comparator CMP, the comparator CMP does not operate when the secondary battery 111 is not charged, the out terminal has a low voltage, and the digital transistor DT1 at the subsequent stage is turned off. Since a voltage drop by the digital transistor DT1 does not occur, the voltage of the EN terminal of the LDO regulator 113 becomes the voltage VBAT of the secondary battery 111.

When the reference voltage Vref is higher than the voltage VBAT during charging of the secondary battery 111, the out terminal of the comparator CMP becomes 5 V, so that the digital transistor DT1 is turned on. As a result, since the EN terminal becomes the ground potential, the LDO regulator 113 stops the voltage supply from the OUT terminal.

On the other hand, when the reference voltage Vref is equal to or lower than the voltage VBAT during charging of the secondary battery 111, the out terminal of the comparator CMP is at the ground potential, so that the digital transistor DT1 is turned off. Here, the reference voltage Vref is generated by dividing a DC voltage of 5 V supplied from an external power supply via the USB connector 108 by the voltage dividing circuit 117 including, for example, two resistors. The reference voltage Vref is set to be equal to or higher than a threshold voltage V_{first-charge} at which the charging IC 110 switches from low-speed charging to fast charging. With such a voltage setting, the output of the LDO regulator 113 is reliably turned off when the voltage VBAT of the secondary battery 111 is lower than the threshold voltage V_{first-charge} of fast charging.

Normally, since the threshold voltage V_{first-charge} for fast charging is higher than the threshold voltage V_{LDO_ON} of the EN terminal of the LDO regulator 113, when the voltage VBAT of the secondary battery 111 becomes higher than the reference voltage Vref (>V_{first-charge}), VBAT, that is, a voltage exceeding V_{LDO_ON} is connected with the EN terminal, and the output of the LDO regulator 113 is turned on. In this manner, since the output of the LDO regulator 113 is turned off during low-speed charging of the secondary battery 111, no charging failure of the secondary battery 111 occurs. Since the reference voltage Vref can be set to an arbitrary value higher than the threshold voltage V_{first-charge} for fast charging as described above, the output of the LDO regulator 113 can be turned on/off with the value of the reference voltage Vref as a boundary.

### Fourth Embodiment

Next, a fourth embodiment of the present invention will be described. The present embodiment describes a specific example of the third embodiment. The outer shape, the mounted sensor type, the basic design, and the function of a wearable sensor terminal of the present embodiment are the same as those of the conventional wearable sensor terminal illustrated in FIGS. 7A and 7B. The configuration of the power supply unit is as illustrated in FIG. 4.

In the present embodiment, the electric resistance of RE is used as the resistor R1 of the power supply unit. As described in the third embodiment, since an external power supply is connected with the Vdd terminal of the comparator CMP, the comparator CMP does not operate when the secondary battery 111 is not charged. Accordingly, since the digital transistor DT1 is turned off when the secondary battery 111 is not charged, the voltage of the EN terminal of the LDO regulator 113 becomes the voltage VBAT of the secondary battery 111.

In the present embodiment, the DC voltage of 5 V supplied from the external power supply via the USB connector 108 is divided by the voltage dividing circuit 117, and the reference voltage Vref=V₁ is inputted to the IN+terminal of the comparator CMP. The voltage VBAT of the secondary battery 111 is inputted to the IN− terminal of the comparator CMP.

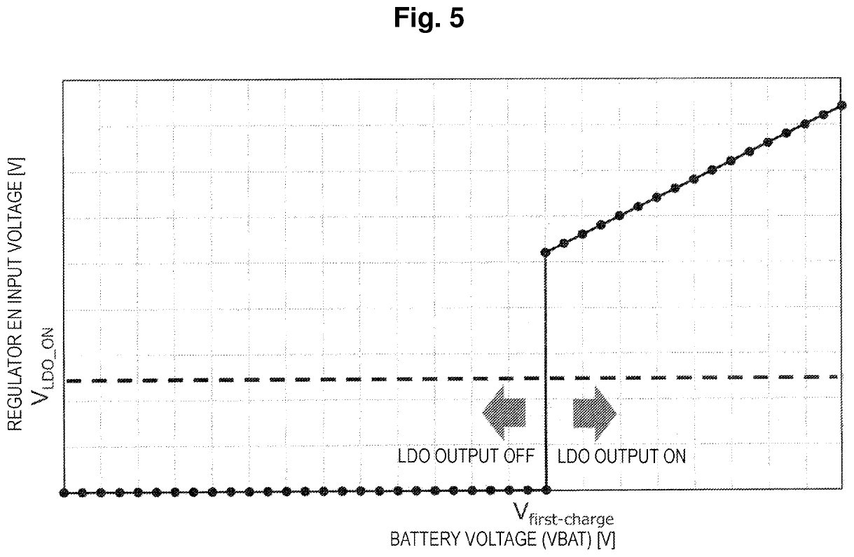

FIG. 5 is a diagram for explaining an operation of the power supply unit of the wearable sensor terminal according to the present embodiment. In this example, Vref=V_{first-charge}=V₁ is satisfied. When the voltage VBAT of the secondary battery 111 is lower than V₁, the digital transistor DT1 is turned on, and the EN terminal becomes the ground potential, and therefore, the LDO regulator 113 stops the voltage supply from the OUT terminal.

When the voltage VBAT of the secondary battery 111 becomes equal to or higher than V₁, the digital transistor DT1 is turned off. That is, the EN terminal of the LDO regulator 113 becomes equal to or higher than V₇ at V₁ or higher at which charging is reliably switched to fast charging. In this manner, ON/OFF of the output of the LDO regulator 113 can be steeply switched. Accordingly, a state where all or most of the charging current of the secondary battery 111 is consumed by the wireless communication module 114 and the secondary battery 111 cannot be charged does not occur.

Regarding the wearable sensor terminal including the protection circuit 116a according to the present embodiment, no charging failure of the secondary battery 111 occurred in all the prepared terminals. In addition, as in the original goal, the communication function can be utilized while charging in a case where the voltage VBAT of the secondary battery 111 is sufficiently high.

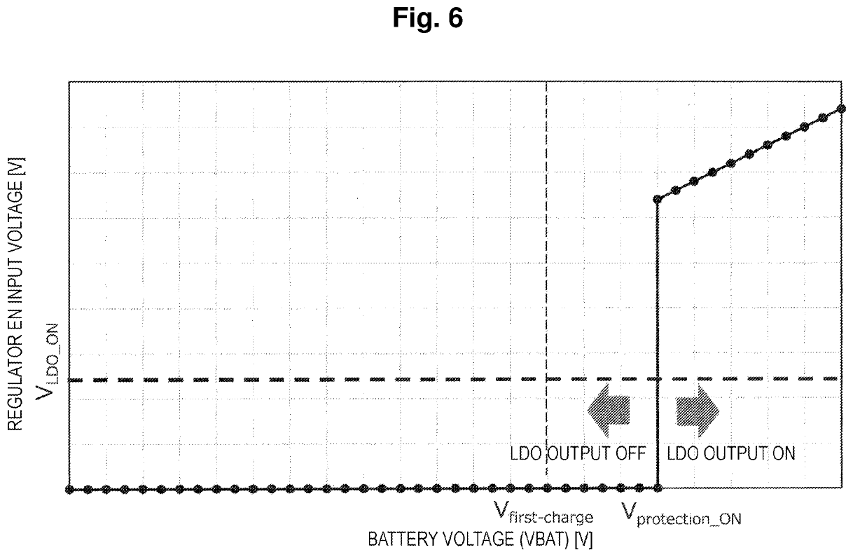

As described above, since the setting of the reference voltage Vref is arbitrary, it is also possible to set the reference voltage Vref not to a value close to the threshold but to a value with a little more margin. FIG. 6 illustrates an operation of the power supply unit in a case where the reference voltage Vref is set to an overdischarging prevention threshold V_{protection_ON}=V₃ of the protection IC 112 (Vref=V_{protection_ON}>V_{first-charge}). In this case, the voltage of the EN terminal of the LDO regulator 113 exceeds the threshold voltage V_{LDO_ON}=V₇ when the voltage VBAT of the secondary battery 111 becomes equal to or higher than V₃. Also in the example of FIG. 6, no charging failure of the secondary battery 111 was observed.

### INDUSTRIAL APPLICABILITY

Embodiments of the present invention can be applied to a wearable sensor terminal.

### REFERENCE SIGNS LIST

101 Resin housing
102 Snap
103 Temperature/humidity sensor
104 Ventilation hole
105 Acceleration/angular velocity sensor
106 LED
107 Power button
108 USB connector
109 Analog front end circuit
110 Charging IC
111 Secondary battery
112 Protection IC
113 LDO regulator
114 Wireless communication module
115 DC/DC converter
116, 116a Protection circuit
117 Voltage dividing circuit
DT1 Digital transistor
CMP Comparator
R1, R2 Resistor

1.-7. (canceled)

8. A wearable sensor terminal comprising:
a sensor configured to measure biological information or environmental information on a vicinity of a living body;
a wireless communication device configured to wirelessly transmit information measured by the sensor to an outside;
a secondary battery;
a charging control device configured to perform fast charging of the secondary battery in a case in which a voltage of the secondary battery is equal to or higher than a first threshold voltage;
a battery protection device configured to prevent overdischarging of the secondary battery;
a regulator configured to convert the voltage of the secondary battery and supply the wireless communication device with the voltage; and
a protection circuit configured to stop supply of a power supply voltage from the regulator to the wireless communication device in a case in which the voltage of the secondary battery is lower than the first threshold voltage at a time of charging.

9. The wearable sensor terminal according to claim 8, further comprising a first resistor having a first end connected with a positive electrode of the secondary battery and an input terminal of the regulator and a second end connected with a control terminal of the regulator, wherein the protection circuit comprises:
a second resistor having a first end connected with a connection point between the first resistor and the control terminal of the regulator; and
a digital transistor having a control input terminal to which a voltage from an external power supply for charging the secondary battery is inputted, a collector terminal connected with a second end of the second resistor, and an emitter terminal connected with ground.

10. The wearable sensor terminal according to claim 9, wherein the regulator is configured to supply the wireless communication device with the power supply voltage in a case in which the voltage of the control terminal is equal to or higher than a second threshold voltage and to stop supply of the power supply voltage to the wireless communication device in a case in which the voltage of the control terminal is equal to or lower than a third threshold voltage that is lower than the second threshold voltage.

11. The wearable sensor terminal according to claim 10, wherein the sensor comprises an electrocardiogram sensor, an acceleration sensor, an angular velocity sensor, or a temperature/humidity sensor.

12. The wearable sensor terminal according to claim 8, further comprising a resistor having a first end connected with a positive electrode of the secondary battery and an input terminal of the regulator and a second end connected with a control terminal of the regulator, wherein the protection circuit comprises:
a comparator configured to compare a reference voltage with a voltage of the secondary battery; and
a digital transistor having a control input terminal to which an output voltage of the comparator is inputted, a collector terminal connected with a connection point between the resistor and the control terminal of the regulator, and an emitter terminal connected with ground.

13. The wearable sensor terminal according to claim 12, wherein the protection circuit further comprises a voltage dividing circuit configured to generate the reference voltage from an external power supply for charging the secondary battery.

14. The wearable sensor terminal according to claim 13, wherein the regulator is configured to supply the wireless communication device with the power supply voltage in a case in which the voltage of the control terminal is equal to or higher than a second threshold voltage and to stop supply of the power supply voltage to the wireless communication device in a case in which the voltage of the control terminal is equal to or lower than a third threshold voltage that is lower than the second threshold voltage.

15. The wearable sensor terminal according to claim 14, wherein the sensor comprises an electrocardiogram sensor, an acceleration sensor, an angular velocity sensor, or a temperature/humidity sensor.

16. The wearable sensor terminal according to claim 14, wherein the wireless communication device comprises a BLE communication module.

17. The wearable sensor terminal according to claim 8, wherein the sensor comprises an electrocardiogram sensor, an acceleration sensor, an angular velocity sensor, or a temperature/humidity sensor as the sensor.

18. The wearable sensor terminal according to claim 8, wherein the wireless communication device comprises a BLE communication module.

19. A system for charging a secondary battery of a wearable sensor terminal, the system comprising:
a sensor configured to measure biological information or environmental information on a vicinity of a living body;
a wireless communication device configured to wirelessly transmit information measured by the sensor to an outside;
a charging control device configured to perform fast charging of the secondary battery in a case in which a voltage of the secondary battery is equal to or higher than a first threshold voltage;
a battery protection device configured to prevent overdischarging of the secondary battery;
a regulator configured to convert the voltage of the secondary battery and supply the wireless communication device with the voltage;
a protection circuit configured to stop supply of a power supply voltage from the regulator to the wireless communication device in a case in which the voltage of the secondary battery is lower than the first threshold voltage at a time of charging; and
a first resistor having a first end connected with a positive electrode of the secondary battery and an input terminal of the regulator and a second end connected with a control terminal of the regulator.

20. The system according to claim 19, wherein the protection circuit comprises:
a second resistor having a first end connected with a connection point between the first resistor and the control terminal of the regulator; and
a digital transistor having a control input terminal to which a voltage from an external power supply for charging the secondary battery is inputted, a collector terminal connected with a second end of the second resistor, and an emitter terminal connected with ground.

21. The system according to claim 20, wherein the regulator is configured to supply the wireless communication device with the power supply voltage in a case in which the voltage of the control terminal is equal to or higher than a second threshold voltage and to stop supply of the power supply voltage to the wireless communication device in a case in which the voltage of the control terminal is equal to or lower than a third threshold voltage that is lower than the second threshold voltage.

22. The system according to claim 21, wherein the sensor comprises an electrocardiogram sensor, an acceleration sensor, an angular velocity sensor, or a temperature/humidity sensor.

23. The system according to claim 19, wherein the protection circuit comprises:
a comparator configured to compare a reference voltage with a voltage of the secondary battery; and
a digital transistor having a control input terminal to which an output voltage of the comparator is inputted, a collector terminal connected with a connection point between the first resistor and the control terminal of the regulator, and an emitter terminal connected with ground.

24. The system according to claim 23, wherein the protection circuit further comprises a voltage dividing circuit configured to generate the reference voltage from an external power supply for charging the secondary battery.

25. The system according to claim 24, wherein the regulator is configured to supply the wireless communication device with the power supply voltage in a case in which the voltage of the control terminal is equal to or higher than a second threshold voltage and to stop supply of the power supply voltage to the wireless communication device in a case in which the voltage of the control terminal is equal to or lower than a third threshold voltage that is lower than the second threshold voltage.

26. The system according to claim 25, wherein:
the sensor comprises an electrocardiogram sensor, an acceleration sensor, an angular velocity sensor, or a temperature/humidity sensor; and
the wireless communication device comprises a BLE communication module.

27. The system according to claim 19, wherein:
the sensor comprises an electrocardiogram sensor, an acceleration sensor, an angular velocity sensor, or a temperature/humidity sensor as the sensor; and
the wireless communication device comprises a BLE communication module.
